(19) United States
(12) Patent Application Publication
(10) Pub. No.: US 2008/0280360 A1
(43) Pub. Date: Nov. 13, 2008

(54) Method for Producing Biomaterial Scaffolds

(75) Inventors: David L. Kaplan, Concord, MA (US); Peter Y. Wong, Brighton, MA (US)

Correspondence Address: DAVID S. RESNICK, NIXON PEABODY LLP, 100 SUMMER STREET, BOSTON, MA, 02110-2131, US

(73) Assignee: TRUSTEES OF TUFTS COLLEGE, Medford, MA (US)

(21) Appl. No.: 11/664,234

(22) PCT Filed: Oct. 12, 2005
(86) PCT No.: PCT/US05/36724

§ 371 Date: Aug. 15, 2007

Related U.S. Application Data
(60) Provisional application No. 60/617,919, filed on Oct. 12, 2004.

(51) Int. Cl. C12N 5/00

(52) U.S. Cl. 435/396

(57) ABSTRACT

The present invention provides a multilayer scaffold for tissue engineering. The scaffold comprises at least a first layer comprised of a polymer having a pattern of microchannels therein; and at least a second layer comprised of a polymer having a pattern of microchannels therein. The first and second layers are joined together (preferably by lamination) and the channels are connected for the circulation of fluid through the layers. The scaffold is coated with bacterial cellulose. The scaffold may further include a mammalian cell.

### CROSS REFERENCED APPLICATIONS

This application claims the benefit under 35 U.S.C. §119(e) of U.S. provisional Patent Application No. 60/617,919, filed Oct. 12, 2004.

### FIELD OF THE INVENTION

The present invention relates to multi-layered scaffolds for tissue engineering that comprise layers of polymer having a defined pattern of microchannels allowing for circulation of fluid throughout the layers and which are coated with bacterial cellulose to support the growth of cells. Methods for preparing these multi-layered scaffolds are also provided.

### BACKGROUND OF THE INVENTION

Tissue engineering is a growing research area that has had numerous advances in understanding the cellular and tissue responses to artificial 2D and 3D scaffolds. However, the 3D scaffolds are typically ill-defined porous structures. Well defined 2D patterning is often explored through microcontact printing, self-assembled monolayers and similar experimental strategies, however, 2D systems are being recognized as non-biologically relevant due to the universal presence of cells in a 3D environment in vivo. Thus, there is a need to explore relationships between cell responses and well-defined 3D matrices to understand the influence of morphology and chemistry on cellular outcomes. Simulating the porous structures in the extracellular matrix environment (ECM) to cells to develop a more fundamental understanding of relationships between the 3D matrices and biological responses should result in better control of cellular growth and tissue outcomes. In order to design better complex 3D scaffold structures, a better understanding of how the cells respond to and grow in specific simple 3D geometries is needed.

Cartilage in limb joints contains a small number of chondrocytes which are spread throughout an ECM and is mainly composed of water, collagen type II and proteoglycans. The cartilage covers the ends in bones at joints in order to provide decreased-friction and to distribute loads [1-3]. The disease ostheoarthritis results in pain and loss of function at the joints [1,4]. Unfortunately, damaged cartilage has limited regenerative capacity; thus, over 1 million patients per year in the United States require some form of treatment. Currently these treatments result in limited pain relief and/or restorative tissue function [5-10]. Tissue engineering has the potential to supply functional cartilage for the repair and regeneration of compromised soft tissues [10,11].

Scaffolds for cartilage are essential in order to support: (1) cell proliferation, (2) maintain their differentiated function, and (3) define the shape of the new growing tissue [1,2]. In attempts to meet these requirements, a variety of scaffold materials have been studied including collagen [13-17], alginate [13,16-18], hyaluronic acid [11,13,16,17], fibrin glue [13,16,17] and chitosan [13,16,17] and synthetic polymers including polyglycolic acid (PGA) [5,7,15-17], poly lactic acid (PLA) [9,16,17], polyvinylalcohol (PVA) [13,16,17,19], polyhydroxyethylmethacrylate (PHEMA) [13,16,17] and polyN-isopropylacrylamide (pNIPAA) [13,16,17]. While many useful insights into cartilage related outcomes have been gained from these studies, there remains a significant gap in cell-matrix understanding and there is a need to move toward more functional and relevant cartilage outcomes. One step toward this goal is to gain improved insight into the 3D matrix-cartilage cell and tissue responses.

There have been a number of attempts at making tissue engineering work for different types of cells [18-20] and specifically creating scaffolds using rapid manufacturing techniques such as microsyringe [21-26] and fused deposited rolling techniques [27,28]. Some initial research has been reported on the effect of scaffold architecture on the composition of tissue engineered cartilage [26]. Two processing techniques, compression-molded/particulate leaching and 3D fiber deposition [25] were used to develop biodegradable scaffolds. The 3D fiber deposition technique produces a square mesh of cylindrical fibers with a large porosity and pore accesibility. Results indicated that in fact, the fiber mesh creates an environment in vivo that enhances cartilaginous matrix deposition. However, the technique does not appear to allow for great control of geometry within the scaffold since it is based on a network of cylindrical fibers. Additional techniques are needed.

### SUMMARY OF THE INVENTION

The present invention provides a multilayer scaffold for tissue engineering. The scaffold comprises at least a first layer comprised of a polymer having a pattern of microchannels therein; and at least a second layer comprised of a polymer having a pattern of microchannels therein. The first and second layers are joined together (preferably by lamination) and the channels are connected for the circulation of fluid through the layers. The scaffold is coated with bacterial cellulose. The scaffold may further include a mammalian cell.

Any polymer can be used in making the scaffold of the invention, including, but not limited to, proteins such as collagen, silk, gelatin, and elastin (or genetic variants thereof); polysaccharides such as amylase, cellulose, amylopectin, starches, pectins, chitosan, chitin, and hyalurinic acid as well as other glycosaminoglycans; synthetic and non-synthetic polymers can be used, such as polycoprolactone (PCL), polylactide-co-glycolide (PLGA), polylactic acid, polyglycolic acid, polyhydroxyalkanoates, polycaprolactone, petroleum derived vinyl polymers (e.g., polyethylene, polyst). Additional polymers useful in making the scaffold of the invention are described in the detailed description.

The polymer can be non-biodegradable or biodegradable and mixtures of polymers can be used.

In one embodiment, the scaffold comprises at least a first layer comprised of polycoprolactone (PCL) and/or polylactide-co-glycolide (PLGA) having a pattern of microchannels therein; and at least a second layer comprised of PCL and/or PLGA having a pattern of microchannels therein. The first and second layers are joined together (preferably by lamination) and the channels are connected for the circulation of fluid through the layers. The scaffold is coated with bacterial cellulose.

The present invention further provides a method for producing a multilayer scaffold. The method comprises providing at least a first layer comprised of a polymer having a defined pattern of microchannels therein; and providing at least a second layer comprised of a polymer having a defined pattern of microchannels therein. The first and second layers are then joined together (preferably by lamination) and the channels are connected for the circulation of fluid through the layers as well as growth of cells. Thereafter, the scaffold is coated with bacterial cellulose, for example by placing the scaffold in a actively growing cellulose producing bacterial culture, e.g., Gluconacetobacter xylinus or mutants or genetic variants thereof.

In one embodiment, a method is provided that comprises providing at least a first layer comprised of polycoprolactone (PCL) and/or polylactide-co-glycolide (PLGA) having a defined pattern of microchannels therein; and providing at least a second layer comprised of PCL and/or PLGA having a defined pattern of microchannels therein. The first and second layers are then joined together (preferably by lamination) and the channels are connected for the circulation of fluid through the layers as well as growth of cells. Thereafter, the scaffold is coated with bacterial cellulose by placing the scaffold in a actively growing cellulose producing bacterial culture, e.g., Gluconacetobacter xylinus or mutants or genetic variants thereof. The scaffold can then be contacted with mammalian cells and placed under appropriate conditions to allow the mammalian cells to grow on the scaffold.

In one preferred embodiment, the microchannels are from 50-500 microns square and millimeter to centimeters thick. The channels are preferably spaced 50-500 microns apart.

As noted above, the scaffold may be used as a matrix for dissociated cells, e.g., chondrocytes or hepatocytes, to create a three-dimensional tissue or organ. Any type of cell can be added to the scaffold for culturing and possible implantation, including cells of the muscular and skeletal systems, such as chondrocytes, fibroblasts, muscle cells and osteocytes, parenchymal cells such as hepatocytes, pancreatic cells (including Islet cells), cells of intestinal origin, and other cells such as nerve cells, bone marrow cells, skin cells, pluripotent cells and stem cells, and combination thereof, either as obtained from donors, from established cell culture lines, or even before or after genetic engineering. Pieces of tissue can also be used, which may provide a number of different cell types in the same structure.

Mammalian cells further include cells selected from the group consisting of hepatocytes, pancreatic Islet cells, fibroblasts, chondrocytes, osteoblasts, exocrine cells, cells of intestinal origin, bile duct cells, parathyroid cells, thyroid cells, cells of the adrenal-hypothalamic-pituitary axis, heart muscle cells, kidney epithelial cells, kidney tubular cells, kidney basement membrane cells, nerve cells, blood vessel cells, cells forming bone and cartilage, smooth muscle cells, skeletal muscle cells, oscular cells, integumentary cells, bone marrow cells, keratinocytes, pluripotent cells and stem cells and combinations thereof.

### BRIEF DESCRIPTION OF FIGURES

FIG. 1 shows a illustration depicting etched silicon with 80 μm square sections with a spacing of 40 μm. The silicon is etched using Deep Reactive Ion Etching (DRIE). In the illustration, the grey region represents the etched portion.

FIG. 2 shows a geometric diagram of the dog bone shape in which the grid pattern etched on the silicon die was embedded in the center (Example 1). The units of the numbers in the diagram are inches.

FIG. 3 shows an isometric view of the epoxy dog-bone master with embedded grid pattern.

FIG. 4 shows an illustration of the mold box that was used to make the polydimethylsiloxane (PDMS) negative (Example 1).

FIGS. 5A through 5C show an illustration of how an individual scaffold layer is formed. FIG. 5A illustrates that melted polycaprolactone (PCL) is poured on top of the PDMS negative. The excess PDMS is then removed by dragging a straight edge across the top of the negative with moderate pressure as shown in FIGS. 5B and 5C.

FIG. 6 shows schematic diagram of an alignment tool that is used to align individual scaffold layers.

FIG. 7 shows an illustration of a scaffold layer and indicates with an x where heated pins were pressed into each of the four corners of the stacked layer within the alignment mold and allowed to cool.

FIG. 8 shows a graph showing the tensile strength of molded (solid) dog bone versus laminated dog bone made with thin layers of PCL. 18 molded PCL thicknesses (dark diamonds) and 8 laminated thicknesses (grey squares), x-axis, were tested for their fracture strength, y-axis.

FIG. 9 shows a microscopic image of a single layer of PCL, and indicates the consistent pore size of the layer prior to exposure to bacterial growth.

FIG. 10 shows a microscopic image of a single layer of PCL after exposure to bacterial growth for 7 days showing that the PCL layer is fully encapsulated with bacterial cellulose.

FIG. 11 shows a microscopic image of a 20 layer PCL scaffold prior to exposure to bacterial growth.

FIG. 12 shows a microscopic image of a 20 layer PCL scaffold after exposure to bacterial growth for 7 days showing that bacterial cellulose is present throughout the layers. the PCL layer is fully encapsulated with bacterial cellulose.

FIG. 13 shows an illustration of a wafer layout of silicon master using Deep Reactive Ion Etching (DRIE). In the illustration, the grey square region represents the etched portion.

### DETAILED DESCRIPTION

The present provides multi-layered scaffolds for tissue engineering that comprise layers of polymer having a defined pattern of microchannels allowing for circulation of fluid throughout the layers and which are coated with bacterial cellulose to support the growth of cells.

The invention also provides methods for producing multi-layered scaffolds. The method comprises a) providing at least a first layer comprised of a polymer having a defined pattern of microchannels therein; b) providing at least a second layer comprised of a polymer having a defined pattern of microchannels therein; c) joining the first and second layer such that channels are connected for the circulation of fluid through the layers; and d) placing the joined layers in a growing bacterial culture for a sufficient period of time to allow the layers to be coated with bacterial cellulose. The scaffold can further be contacted with mammalian cells and placed under appropriate conditions to allow the mammalian cells to proliferate on the scaffold.

### Layered Scaffolds

Any technique for producing layered scaffolds with defined channels that are known to those skilled in the art can be used in methods of the invention. Techniques for producing molds and scaffold layers using microfabricated assembly technology (e.g. micromachined wafer technology, thick photoresist processes, hot embossing) and soft lithography are set forth in the examples below and in, for example, WO 02/053193 and WO 03/004254.

In one preferred embodiment, soft lithography techniques are applied to thermal lamination (e.g. See Example 1). Soft lithography is the use of polymer molds in conjunction with photolithography. The first step in the production of biopolymer scaffolds by any of the soft lithography methods of the invention is the production of a silicon template that allows the fabrication of the master mold (e.g. elastomer mold (negative)). Means of producing silicone templates are known to those in the art. The silicon template is then etched to make a silicone master with user defined properties, e.g. a grid pattern, using a photolithographic mask. Means for etching silicone templates are also well known to those skilled in the art.

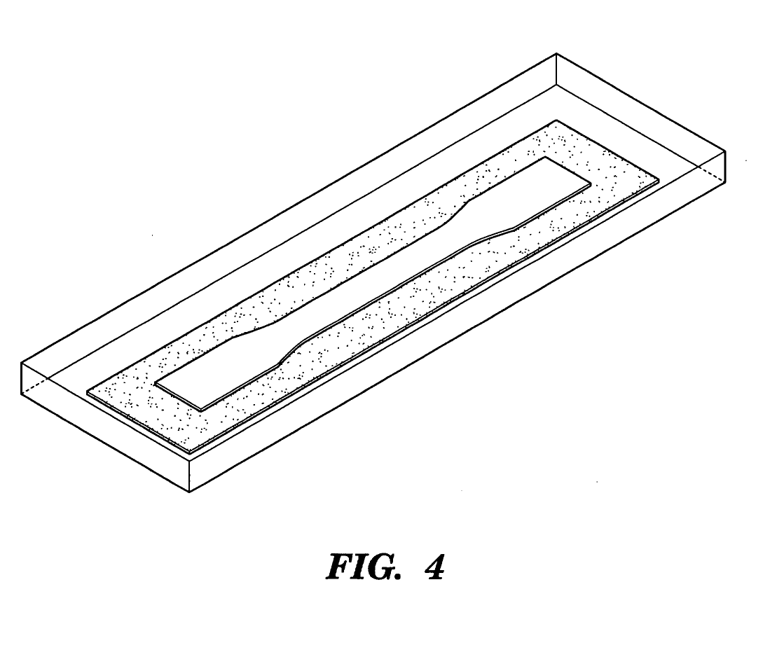

Polymer molds are then cast from the fabricated silicon master (e.g. as described in the examples, or in WO 03/004254). In one preferred embodiment the polymer mold is made by mixing and pouring liquid polymer (e.g. PDMS) onto the silicone template (See FIG. 4). Dependent on what polymer is used for the mold, the polymer may require the presence of a solvent for mold preparation. Preferably the mixture is degassed under vacuum to remove any bubbles that may have been introduced. Further, when a solvent is used, preferably the template/mold is baked to drive away solvent. After casting of the polymer mold (e.g. elastomer mold), the polymer is allowed to cure and can be gently peeled away from the silicone master forming a master mold. The mold can then be washed with 70% ethanol and sonicated prior to use.

Once the master mold is obtained, casting of the polymer scaffold layers can be performed using any known means, e.g. a micromolding method where the polymer is cast on the mold under vacuum; a microfluidic method, where the polymer mold is sealed onto a desired substrate (e.g. glass, plastic) and the polymer solution for casting is forced to flow through the channels by applying negative pressure; a spin-coating method, where the polymer solution is spin coated onto the mold to allow the fabrication of thin membranes of non-uniform in height; or simply by pouring polymer solution over the top of the master mold. The selection of the method to be used depends on a number of factors including equipment available and the skills of the user.

In one preferred embodiment, hot liquid polymer is poured over the top surface of the master mold (e.g. elastomer negative) and a straight edge is dragged across the top of the master mold with moderate pressure to remove excess material (See example 1). After cooling at room temperature the master polymer mold is peeled away from the solidified polymer scaffold layer.

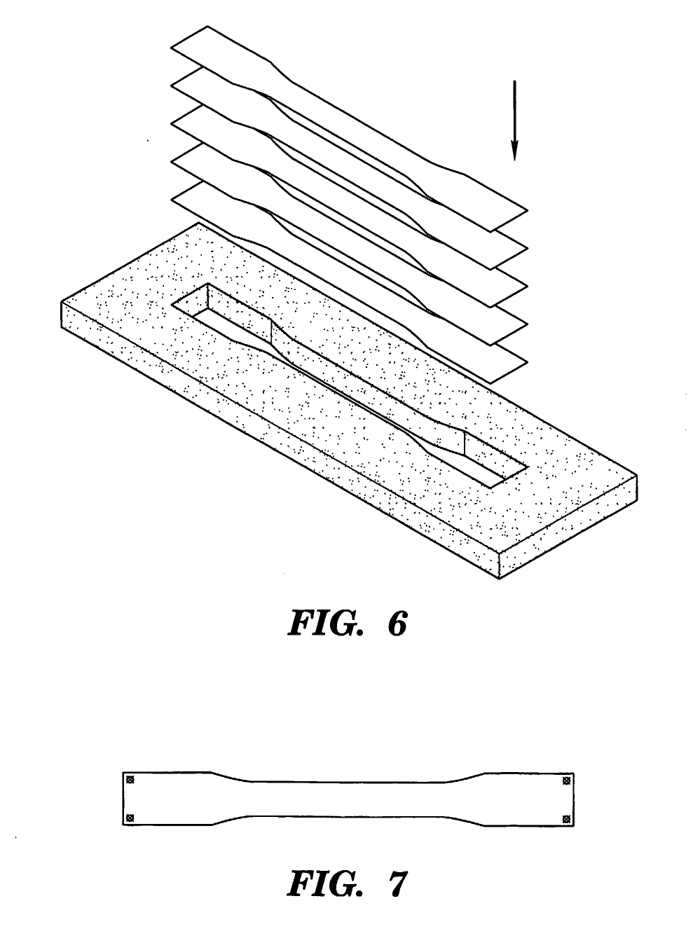

Each individual scaffold layer is then layered on top of one another. In one preferred embodiment, individual scaffold layers are stacked together in an alignment mold (e.g. FIG. 7). Heated pins can be pressed into each of the four corners of the stack to ensure alignment. The use of an alignment mold ensures development of well defined channels with in the scaffold. Any number of layers can be stacked.

The layers are then adhered to each other by known means, e.g. with PDMS, application of a thin layer of solvent can act as binder; or by applying a mechanical load to a set of PLGA membranes stacked together and heating for 10 minutes at 60 degrees C.; or by thermal lamination in a standard laminator (e.g. for PCL layers, See Example 1).

### Polymers

Any polymer can be used to prepare the scaffold of the invention, including, but not limited to, proteins such as collagen, silk, gelatin, and elastin (or genetic variants thereof); polysaccharides such as cellulose, amylase, amylopectin, starches, pectins, chitosan, chitin, and hyalurinic acid as well as other glycosaminoglycans; synthetic and non-synthetic polymers can be used such as polycoprolactone (PCL), polylactide-co-glycolide (PLGA), polylactic acid, polyglycolic acid, polyhydroxyalkanoates, polycaprolactone, petroleum derived vinyl polymers (e.g., polyethylene, polyst).

The polymer can be non-biodegradable or biodegradable and mixtures and combinations of polymers can be used. Further examples of polymers are described below.

Among the materials that can be used to create the scaffolds are polymers made of representative synthetic polymer blocks, including polyphosphazenes, poly(vinyl alcohols), polyamides, polyester amides, poly(amino acid)s, synthetic, poly(amino acids), polyanhydrides (such as polyanhydride co-polymers of fumaric and sebacic acid (poly(FA:SA)), polycarbonates (U.S. Pat. Nos. 5,099,060 and 5,198,507), polyarylates (U.S. Pat. No. 5,216,115), polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. See, U.S. Pat. No. 6,160,084; or The Polymer Handbook, 3rd edition (Wiley, N.Y., 1989). The utility of a polymer as a tissue engineering substrate is primarily dependent upon whether it can be readily fabricated into a three-dimensional scaffold.

Examples of suitable polyacrylates include poly(methyl methacrylate) (PMMA), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecylacrylate). Other examples of suitable polymers include the polyethylene oxide/polyethylene terephthalate disclosed by Reed et al., Trans. Am. Soc. Artif: Intern. Organs, 109 (1977); bisphenol-A based polyphosphoesters, including poly(bisphenol-A phenylphosphate), poly(bisphenol-A ethylphosphate), poly(bisphenol-A ethylphosphonate), poly(bisphenol-A phenylphosphonate), poly[bis(2-ethoxy) hydrophosphonic terephthalate], and copolymers of bisphenol-A based poly(phosphoesters) (see, U.S. Pat. No. 5,686,091); and polymers of tyrosine-derived diphenol compounds. Methods for preparing the tyrosine-derived diphenol monomers are disclosed in U.S. Pat. Nos. 5,587,507 and 5,670,602.

When the scaffold is intended for implantation, the polymer should be selected for biocompatibility at the time of implant, any degradation products should also be biocompatible. Relatively high rigidity is advantageous so that the scaffold can withstand the contractile forces exerted by cells growing within the scaffold.

Also important are the thermal properties, especially the glass transition temperature (Tg) which must be high enough so that the network of pores in the scaffold does not collapse upon solvent removal.

A biocompatible degradable polymer and its degradation products are non-toxic toward the recipient. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refer to cleavage of the polymer chain, such that the molecular weight stays approximately constant at the oligomer level and particles of polymer remain following degradation. The term “completely degrade” refers to cleavage of the polymer at the molecular level such that there is essentially complete mass loss. The term “degrade” as used herein includes “completely degrade” unless otherwise indicated. PLGA, as well as PLA and PGA have been used to make biodegradable implants drug delivery. See, U.S. Pat. No. 6,183,781 and references cited therein. Biodegradable materials have been developed for use as implantable prostheses, as pastes, and as templates around which the body can regenerate various types of tissue. Polymers that are both biocompatible and resorbable in vivo are known in the art as alternatives to autogenic or allogenic substitutes.

Representative synthetic biodegradable polymer segments or polymers include polyhydroxy acids, such as polylactides (PLA), polyglycolides (PGA), and copolymers thereof; poly(ethylene terephthalate); poly(hydroxybutyric acid); poly(hydroxyvaleric acid); poly[lactide-co-(-caprolactone)]; poly[glycolide-co-([pound]-caprolactone)]; polycarbonates, poly(pseudo amino acids); poly(amino acids); poly(hydroxyalkanoate)s; polyanhydrides; polyortho esters; and blends and copolymers thereof. These bioerodable polymers also include polyacetals, polyeyanoaerylates, poly(ether ester)s, poly(dioxanone)s, poly(alkylene alkylate)s, copolymers of poly(ethylene glycol) and poly(ortho ester), degradable polyurethanes and copolymers and blends thereof. Also included are non-bioerodable polymers such as polyacrylates, ethylene-vinyl acetate copolymers, acyl-substituted cellulose acetates, non-degradable polyurethanes, polystyrenes, polyvinyl chloride, polyvinyl fluoride, poly(vinylimidazole), ehlorosulfonate polyolefins, and polyethylene oxide.

Any suitable blends or copolymers of these materials can also be used. Solvent/nonsolvent systems suitable for a given polymer can be determined via routine experimentation. See, U.S. Pat. No. 6,183,781.

Rapidly biodgradable polymers such as polylactide-co-glycolides, polyanhydrides, and polyorthoesters, which have carboxylic groups exposed on the external surface as the smooth surface of the polymer erodes, can also be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Particularly useful for this invention are polyesters in the polylactide(PLA)/polyglycolide(PLG) family. These polymers have received a great deal of attention in the drug delivery and tissue regeneration areas. They have been in use for over 20 years in surgical sutures, are Food and Drug Administration (FDA)-approved and have a long and favorable clinical record. A wide range of physical properties and degradation times can be achieved by varying the monomer ratios in lactide/glycolide copolymers. Poly-L-lactic acid (PLLA) and poly-glycolic acid (PGA) exhibit a high degree of crystallinity and degrade relatively slowly, while copolymers of PLLA and PGA, PLGAs, are amorphous and rapidly degraded.

A preferred polymeric material that can be used to create the scaffolds is poly(D,L-lactide-co-glyeolide (polylaetide-co-glyeolide; PLGA). PLGA is biocompatible and biodegradable. Particularly useful for the practice of the invention, PLGA can be stacked or bonded. PLGA can also be of varying and controllable porosity. Moreover, PLGA can be cast, stamped, or embossed and direct etching of PLGA is possible.

Another preferred polymer for use in the invention is polycoprolactone (PCL).

Methods for making biodegradable polymers in desired shapes are known in the art. See, U.S. Pat. No. 6,165,486. Suitable solvents for forming the polymer solution include methylene chloride, acetone, ethyl acetate, methyl acetate, tetrahydrofuran and chloroform. For example, a solution of PLGA can readily be prepared in methylene chloride. Solvent casting is one of the most widely used processes for fabricating scaffolds of degradable polymers. See, U.S. Pat. No. 6,103,255; U.S. Pat. No. 5,686,091; U.S. Pat. No. 5,723,508; U.S. Pat. No. 5,514,378; Mikos et al., Polymer 35: 1068-77, (1994); de Groot et al., Colloid Polym. Sci. 268: 1073-81 (1991); and Laurencin et al., J. Biomed. Mater. Res. 30: 133-8 (1996)).

Biocompatible, non-biodegradable polymers can also be used in the invention for constructing artificial organs where the scaffold is not intended to degrade following implantation. Examples of non-biodegradable polymer segments or polymers include ethylene vinyl acetate, poly(meth) acrylic acid, polyamides, polyethylene, polypropylene, polystyrene, polyvinyl chloride, polyvinylphenol, and copolymers and mixtures thereof.

A preferred non-biodegradable polymeric material that can be used to create the scaffolds is polydimethylsiloxane (PDMS). Silicones are polymeric organosilicon compounds. The repeating (SiO(CH3)2) unit is the monomer of which the polymer PDMS is composed. There are six classes of silicone products: fluids, lubricants, elastomers (rubbers), resins, emulsions, compounds and fluids. Each of these classes depends upon the number of the monomeric units and the degree to which the chains are crosslinked.

Elastomers are used for the polymeric scaffold material in this invention (See U.S. Pat. No. 5,776,748). PDMS is a common structural material used in biomedical applications (Hong J W, IEEE-EMBS Cony Microtechnol. In Medicine and Biology, 407 (2000)).

Like PLGA, PDMS can be stacked or bonded; can be made of varying and controllable porosity; and can be cast, stamped, embossed or etched.

Another non-biodegradable polymeric material that can be used to create the scaffolds is polymethylmethacrylate (PMMA).

Moreover, advances in polymer chemistry can aid in biologic tasks of adhesion and gene expression. For example polymers modified with specific adhesive peptides or proteins. Some alternative polymer systems are described in WO 02/053193 and WO03/004254.

Coating the Layered Scaffold with Bacterial Cellulose

In methods of the invention, the layered scaffold is coated with bacterial cellulose. This can be accomplished by growing bacteria in the presence of the scaffold, or by coating the scaffold manually with bacterial cellulose that has been isolated form its source.

Bacterial cellulose is a cellulose produced by bacteria. Plant cellulose and bacterial cellulose have the same chemical structure, but different physical and chemical properties, the diameter of bacterial cellulose is about 1/100 of that of plant cellulose.

Any bacterium that produces bacterial cellulose can be used in methods of the invention. In one preferred embodiment Acetobacteria, such as Gluconacetobacter xylinus are used (see general review by Ross, P. et al. Microbiol. Rev. 1991, 55: 35-50). Peudomonas fluorescens can also be used, see PCT Publication WO-A 2002004526. Alternatively, E. coli that has been genetically engineered to produce bacterial cellulose can be used, all genes responsible for bacterial cellulose synthesis have been cloned. Wong et al. and Ben Bassat et al. have shown that the biosynthesis of bacterial cellulose is related to the activity of four genes, best (2261 base pairs), bcsB (2405 base pairs), besC (3956 base pairs) and bcsD (467 base pairs), which form the cellulose synthase operon that is 9217 base pairs long (Wong H. C., et al. (1990) Genetic organization of the cellulose synthase operon in Acetobacterxylinum. Proc. Natl. Acad. Sci. USA 87, 8130-8134; Ben-Bassat A. et al. (1993) Methods and nucleic acid sequences for expression of the cellulose synthase operon. U.S. Pat. No. 5,268,274). Nakai T. et al, Biochem Biophys Res Commun. 2002 Jul. 12; 295(2):458-62, has further shown that ORF2 gene is involved in the construction of high-order structure of bacterial cellulose.

Medium used for bacterial growth and production of cellulose are well known to those skilled in the art. In one embodiment, the media used for growth comprises 10 g/l Bactopeptone (Difco), 10 g/l yeast extract (Fisher), 4 mM KH₂PO₄ (Sigma), 6 mM K₂HPO₄ (Sigma) and 20 g/l D-glucose dissolved in deionized water (DI); pH 5.1-5.2. Production of bacterial cellulose by Acetobacter xylinum BPR2001 using molasses medium in a jar fermentor has also been described (Bae S O, Shoda M. Appl Microbiol Biotechnol. 2005 April; 67(1):45-51. Epub 2004 Aug. 25.). Other mediums used for producing bacterial cellulose are described in WO2005003366 entitled Method for the Production of Bacterial Cellulose.

The bacteria is grown in the presence of the scaffold for a sufficient time to allow for coating of the scaffold with the excreted cellulose. This can be from 1 day to 1 week to several weeks, depending on cellulose production and the concentration of bacteria. The presence of cellulose coating can be monitored by microscopy by using for example, fluorescently labeled DTAF (Sigma) which binds to exposed hydroxyl groups on cellulose chains. Preferably the scaffold has a uniform coating of cellulose. In one embodiment, 25%-45% of the scaffold surface area is coated with cellulose, preferably 50%-75% of the scaffold surface area is coated with cellulose, more preferably 70%-95%, even more preferably up to 99%-100% of the scaffold is coated.

When the scaffold is grown in the presence of bacteria the produce cellulose, in certain embodiments, it is important to remove the bacteria after sufficient coating. Bacteria can be removed by simply washing the scaffold multiple times in a suitable media, e.g. water, with or without centrifugation between washings. Alternatively, the bacterial cells can be enzymatically digested prior to washing. In one embodiment, osmotic stress is used to burst the bacteria prior to washing.

The successful removal of bacteria can be monitored visually using a microscope or by other means known in the art.

Methods of the invention further comprise contacting the layered-coated scaffold with mammalian cells under appropriate conditions to allow the mammalian cells to proliferate on the scaffold. Any mammalian cell can be added to the scaffold of the invention including, but not limited to, hepatocytes, pancreatic Islet cells, fibroblasts, chondrocytes, osteoblasts, exocrine cells, cells of intestinal origin, bile duct cells, parathyroid cells, thyroid cells, cells of the adrenal-hypothalamic-pituitary axis, heart muscle cells, kidney epithelial cells, kidney tubular cells, kidney basement membrane cells, nerve cells, blood vessel cells, cells forming bone and cartilage, smooth muscle cells, skeletal muscle cells, oscular cells, integumentary cells, bone marrow cells, keratinocytes, pluripotent cells and stem cells and combinations thereof.

The scaffold may be used as a matrix for dissociated cells, e.g., chondrocytes or hepatocytes, to create a three-dimensional tissue or organ. Any type of cell can be added to the scaffold for culturing and possible implantation, including cells of the muscular and skeletal systems, such as chondrocytes, fibroblasts, muscle cells and osteocytesi parenchymal cells such as hepatocytes, pancreatic cells (including Islet cells), cells of intestinal origin, and other cells such as nerve cells, bone marrow cells, skin cells, pluripotent cells and stem cells, and combination thereof, either as obtained from donors, from established cell culture lines, or even before or after genetic engineering. Pieces of tissue can also be used, which may provide a number of different cell types in the same structure. Media for these various cell lines are known to those in the art.

In one preferred embodiment, the scaffold is seeded dissociated chondrocytes. The formation of cartilaginous tissue can be monitored by assays well known to those in the art including, but not limited to, histology, immunohistochemistry, and confocal or scanning electron microscopy (Holy et al., J. Biomed. Mater. Res (2003) 65A:447-453). Formation of other tissues (muscle, bone, skin etc.) can also be monitored by these means.

In one preferred embodiment, the scaffolds are seeded with multipotent cells in the presence of media that induces either bone or cartilage formation. Suitable media for the production of cartilage and bone are well known to those skilled in the art.

As used herein, “multipotent” cells have the ability to differentiate into more than one cell type in response to distinct differentiation signals. Examples of multipotent cells include, but are not limited to, bone marrow stromal cells (BMSC) and adult or embryonic stem cells. In a preferred embodiment BMSCs are used. BMSCs are multipotential cells of the bone marrow which can proliferate in an undifferentiated state and with the appropriate extrinsic signals, differentiate into cells of mesenchymal lineage, such as cartilage, bone, or fat (Friedenstein, A. J. 1976. Int Rev Cytol 47:327-359; Friedenstein et al. 1987. Cell Tissue Kinet 20:263-272; Caplan, A. I. 1994. Clin Plast Surg 21:429-435; Mackay et al. 1998. Tissue Eng 4:415-428; Herzog et al. Blood. 2.003 Nov. 15; 102(10):3483-93. Epub 2003 Jul. 31). Using the scaffolds described herein, organized tissue with a predetermined form and structure can be produced either in vitro or in vivo. For example, tissue that is produced ex vivo is functional from the start and can be used as an in vivo implant. Alternatively, the scaffold can be seeded with cells capable of forming tissue (e.g. bone or cartilage) and then implanted as to promote growth in vivo.

All biomaterials of the present intention may be sterilized using conventional sterilization process such as radiation based sterilization (i.e. gamma-ray), chemical based sterilization (ethylene oxide), autoclaving, or other appropriate procedures. Preferably the sterilization process will be with ethylene oxide at a temperature between 52-55° C. for a time of 8 hours or less. After sterilization the biomaterials may be packaged in an appropriate sterilize moisture resistant package for shipment and use in hospitals and other health care facilities.

The following examples are presented in order to more fully illustrate the preferred embodiments of the invention. These examples in no way should be construed as limiting the scope of the invention as defined by the appended claims.

### EXAMPLES

### Example 1

### Layered Scaffolds

1.3.1. Bio-LOM Process (LOM stands for laminated object manufacturing; Bio-LOM refers to the technique which includes lining the scaffold with organic materials, such as bacterial cellulose)

The techniques for soft lithography [37] are applied to thermal lamination.

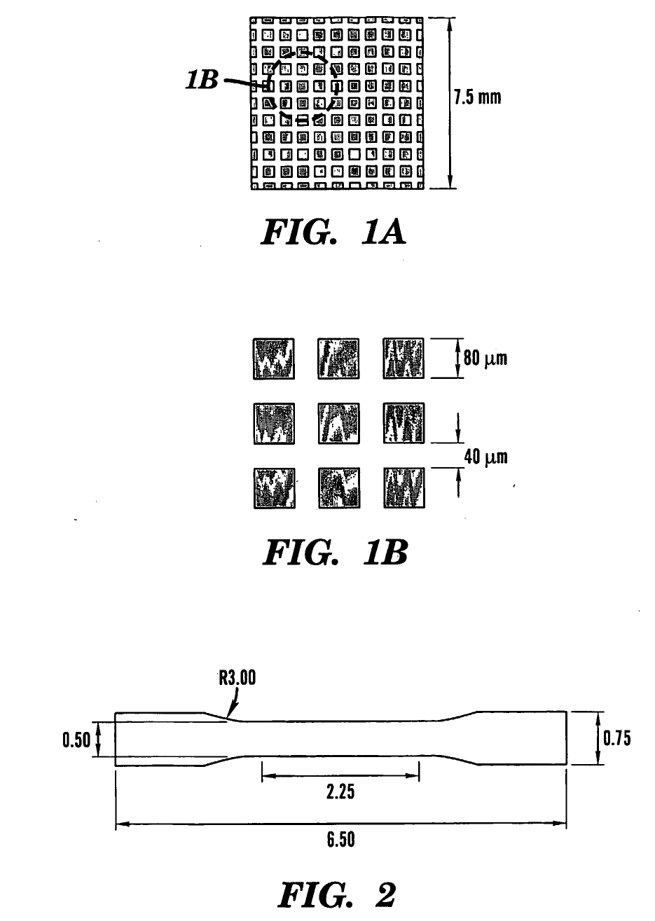

1.3.1.1. Advanced etching was used to make a silicon master of a die with user-defined pattern. As seen in FIG. 1, a grid pattern (80 μm square sections with spacing of 40 μm) was designed using Intellisuite 0 software (Intellisuite Software, Woburn, Mass.). A photolithographic mask of the desired pattern was made by depositing a thin film of chromium onto a flat glass panel (Benchmark Technologies, Lynnfield, Mass.) The mask was sent to a silicon foundry service (MEMS Exchange, Reston, Va.) for Deep Reactive Ion Etching (DRIE) onto a silicon wafer to a vertical depth of 90 μm. Prior to etching, the mask was used to photochemically cure a thin layer of photoresist on the areas of the wafer not to be etched (the white area as seen FIG. 1.) Once the etching was done, the photoresist was dissolved and stripped away from the master.

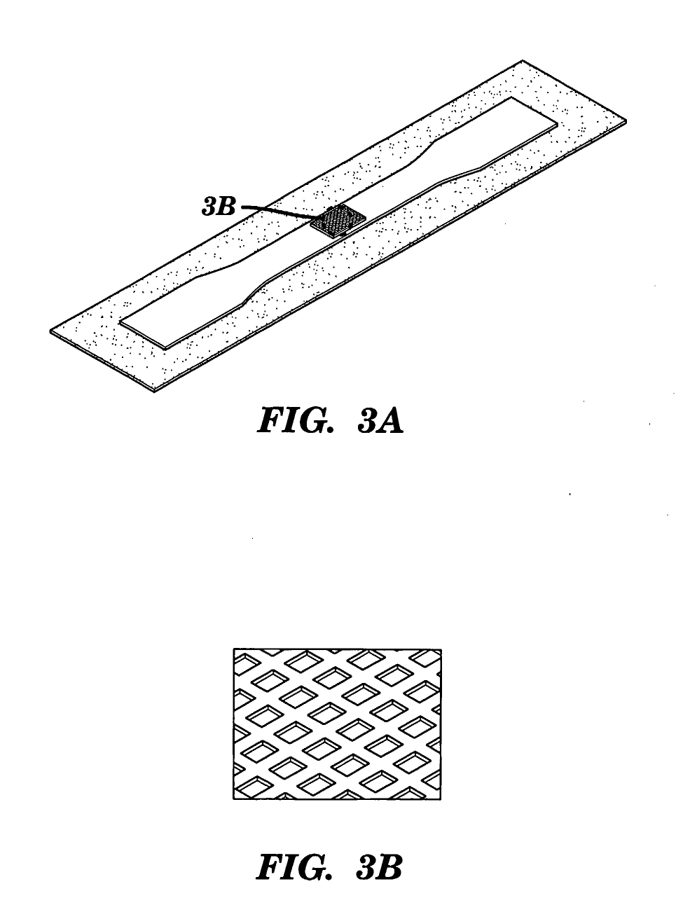

1.3.1.2 Epoxy dog-bone master with embedded grid pattern is fabricated using a multi-step process. To facilitate controlled tensile testing of the scaffold, the grid pattern etched on the silicon die was embedded into the center of a 6.5″ dog-bone geometry. The dog-bone geometry (FIGS. 2 and 3) not only conforms to ASTM and ANSI standards for tensile testing of plastic materials, but also provides alignment for laminating successive layers of the scaffold (see section 1.3.1.5.)

Elastomer negative was made from the epoxy master by mixing and pouring liquid polydimethylsiloxane (PDMS) into a mold box (see FIG. 4). “Sylgard™ 184” (GE Silicones, Wilton, Conn.) base and curing agent is mixed thoroughly at 1:10 weight ratio for 2 minutes. After vacuum degassing of the liquid PDMS for 20 minutes, the PDMS was allowed to cure at room temperature for 24 hours. Once cured, the PDMS negative was gently peeled away from the master.

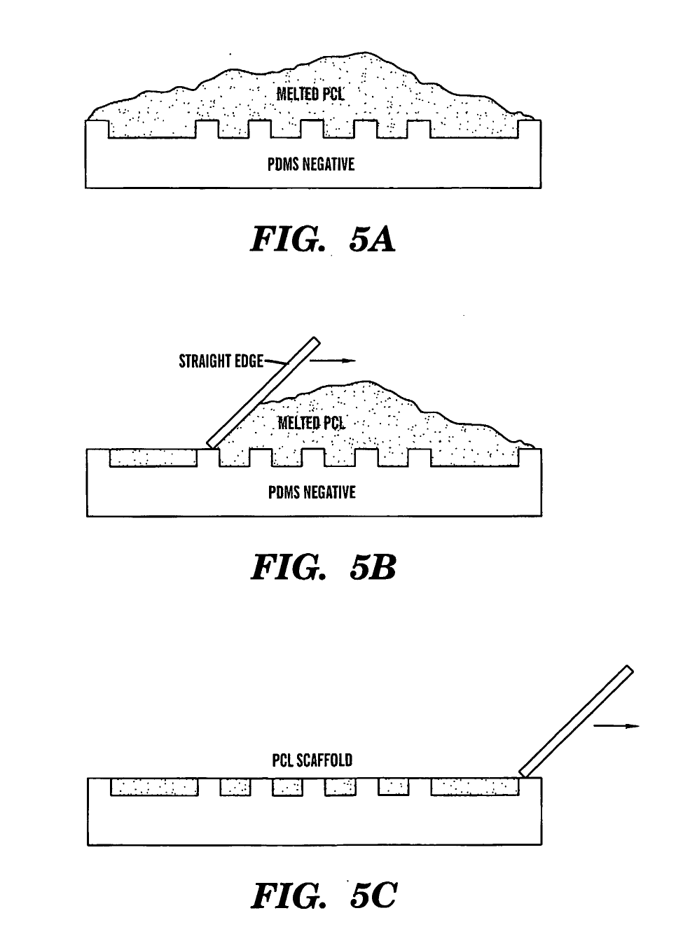

1.3.1.4. Individual scaffold layer was produced by casting polycaprolactone (PCL: Tₘ˜60° C.) that has been heated to 100° C. in an oven. The liquid PCL was poured over the top surface of the PDMS negative (see FIG. 5a). A straight edge, such as the edge of a glass slide or razor blade (see FIG. 5b), is dragged across the top of the negative with moderate pressure to remove excess material (see FIG. 5c). The negative is allowed to cool for 30 minutes at room temperature. The PDMS negative is gently peeled away from the solidified PCL scaffold layer.

Layer Alignment was ensured by stacking several layers together in a mold (see FIG. 6). Heated pins were pressed into each of the four corners of the stack layer and allowed to cool (see FIG. 7). The pins were then removed from the stack that was now assembled with proper alignment.

Thermal lamination of scaffold at a temperature of 75° C. in a standard laminator. The stack of scaffold layers was placed in a sleeve of aluminum foil. The foil was placed into a paper carrier and fed through the laminator. Once the carrier has exited the laminator, the carrier was allowed to cool for 10 minutes at room temperature, and the laminated scaffold layers were extracted from the carrier and foil.

### Tensile Strength of Scaffold Material.

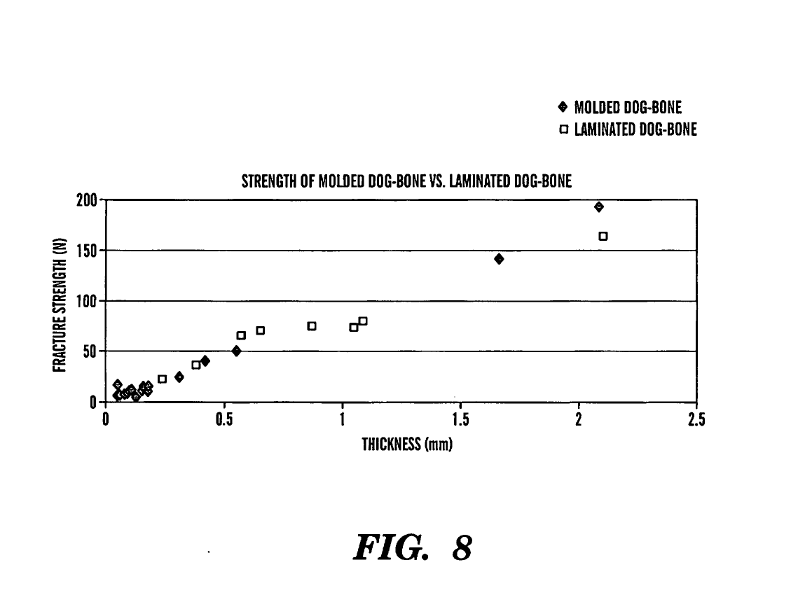

In order to verify that the thermal lamination technique produced solid bonding between layers, a comparision test for mechanical strength was conducted between solid bars of PCL molded in dog-bone shapes and a laminated dog-bone made with thin layers of PCL. There were 18 molded PCL thicknesses and 8 laminated thicknesses ranging from 0.05 mm to 2.05 mm. The tensile test results presented in FIG. 8 shows the good coorelation between the solid and laminated samples. This good bonding was important to consistent cellular response as well as mechanical strength.

### Production of Bacterial Cellulose (BC)

Gluconacetobacter xylinus (=Acetobacter xylinum(ATCC 10245) was purchased from the American Type Culture Collection and grown in 10 g/l Bactopeptone (Difco), 10 g/l yeast extract (Fisher), 4 mM KH₂PO₄ (Sigma), 6 mM K₂HPO₄ (Sigma) and 20 g/l D-glucose dissolved in deionized water (DI). The pH of the medium was adjusted to 5.1-5.2. Media was inoculated with culture and grown in the presence of the PCL scaffolds at 30° C. for 7 days.

### BC Growth in Scaffolds

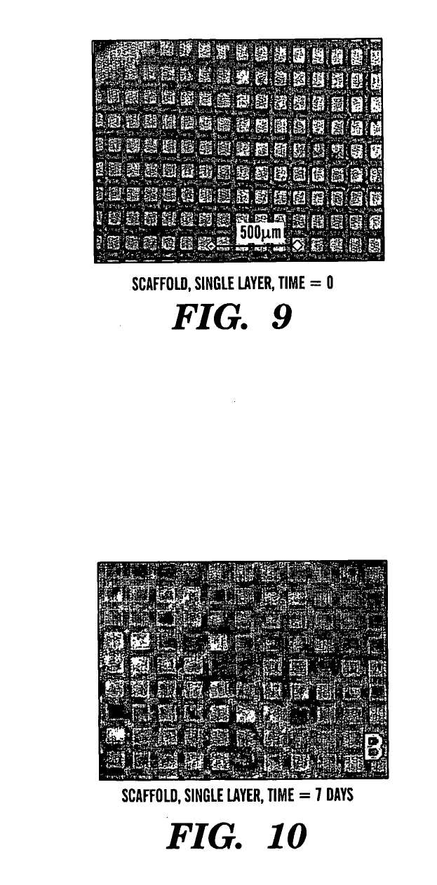

A single layer of PCL as seen in FIG. 9 has consistent pore size. This PCL sample was subjected to BC growth as described in the previous section. The resultant cellular growth is seen in FIG. 10. Observations using an optical microscope show that the scaffold was fully encapsulated with BC.

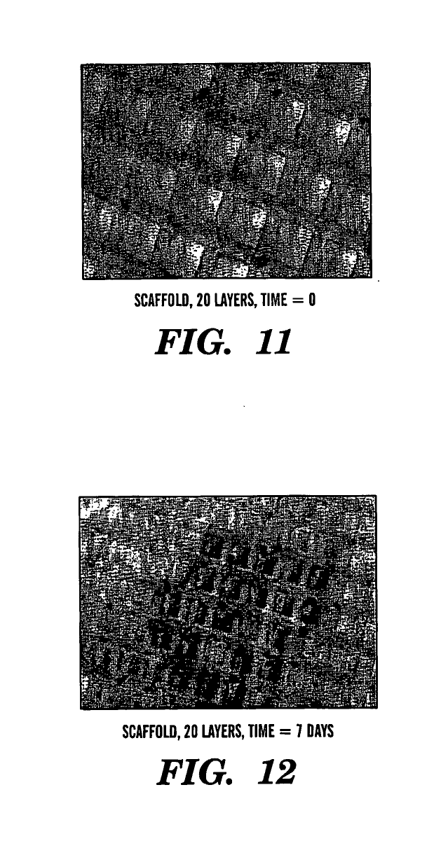

The next experiment was conducted on a 20 layer scaffold. The scaffold before growth is shown in FIG. 11. The pores did not line up consistently for this 20 layer sample; however, efforts to line up the pores more evenly should be possible with some minor adjustments to the alignment tool. Nevertheless, in the 20 layer sample there was BC growth throughout the scaffold interior (FIG. 12).

A chemical analysis of the BC in the scaffold is described in section 2.2.1 and testing for mechanical strength (described in section 2.2.2) of the composite structure is conducted for wet and dry state. The BC growth was our model system to verify that the PCL scaffolds can support good cell response.

### Example 2

### Simplified Method for Scaffold Fabrication

2.1.1. Improved Bio-LOM process

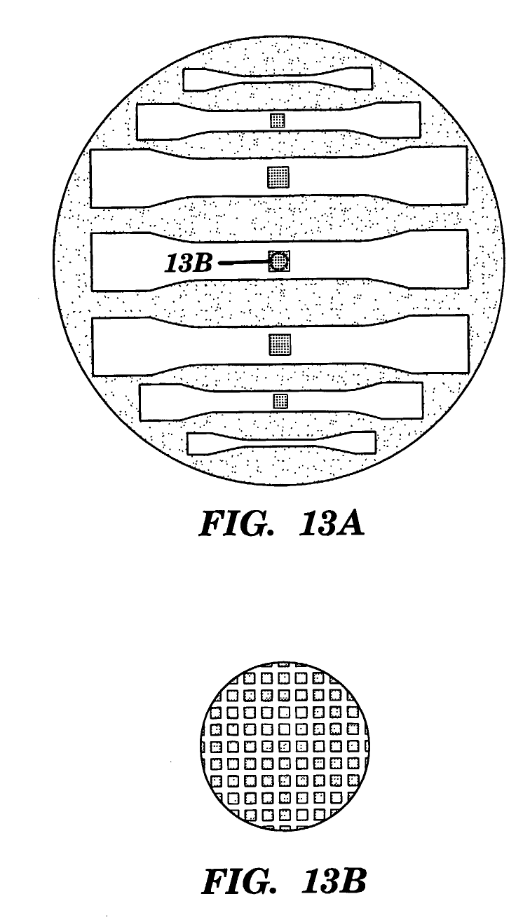

A simpler process in developing a silicon master dog-bone with embedded, grid pattern is described that can be used material such as PLGA. The Bio-LOM processing steps after section 1.3.1.2 are to remain the same. In the new process, a single photolithographic mask of the desired complete pattern for each layer is made with the dog-bone shape instead of embedding the smaller square sections. This allows for less user-error in the process. A number of dog-bones with etched pattern can be fabricated on a wafer (see FIG. 13).

### 2.2 Experimental Method for Growth and Testing

The well-defined scaffolds are characterized, introduced to chondrocytes and appropriate media, and then analyzed chemically and mechanically.

### 2.2.1. Characterization

Scanning Electron Microscopy (SEM). Zeiss DSM 940A is used to study the surface morphology of the materials before and after chondrocyte growth. Phase-contrast microscopy (Axiovert S100 from Zeiss) is used to study cell growth in 3D. Confocal microscopy BioRad MRC1024 equipped with fiber coupled ArKr laser is used to study the morphology and porosity of the materials in wet state as we have previously reported [29]. Filters were chosen with regard to the emission wave length of the dye. [λex=495 nm and λem=516] The wet bacterial cellulose samples were fluorescently labeled using DTAF (5-([4,6-Dichlorotriazin-2-yl]amino)fluorescein hydrochloride) [Sigma], which binds to exposed hydroxyl groups on the cellulose chains.

### 2.2.3. Cell Responses

Scaffold samples are sterilized by 70% ethanol and transferred to a large well tissue culture plate in triplicate. One ml DMEM (Gibco) is added to each well to soak the samples before cell seeding. Primary bovine chondrocytes (passage number 6 and 95% viability) are obtained by enzymatic digestion of full-thickness articular cartilage harvested from the femoropatellar grooves of 2- to 3-week-old bovine calves, and seeded at a concentration of 25,000 cells per well. One ml media is added to each well and plates are incubated for 8 days at 37° C., 5% CO₂. Media is changed every three days and images captured each day. The cell culture media consists of Dulbecco's modified Eagle's medium (DMEM) [Gibco] containing 1% penicillin-streptomycin (P/S), 0.2% fungizone, 1% N²-hydroxyethylpiperazine-N′-2-ethane sulfonic acid (HEPES) and 10% fetal bovine serum (FBS) [Gibco] plus 1% non-essential amino acids (NEAA), 0.1M proline and 50 μg/ml L-ascorbic acid [Sigma].

### 2.3.4 Biochemical Analyses

2.3.4.1 DNA analysis, n=4 constructs per group and time point (after 6 hours (initial conditions, 1,2 and 4 weeks) are desintegrated using steel balls and a Minibead-beadbeater (Biospec, Bartlesville, Okla.). DNA content is measured fluorometrically using the PicoGreen assay (Molecular Probes, Eugene, Oreg.), according to the protocol of the manufacturer (excitation wavelength of 480 nm; emission wavelength of 528 nm).

2.3.4.2 Metabolic activity of the cells can be assessed by the MTT assay. For each group and time point, n=4 constructs are transferred into a 2 ml plastic tube. The reagent is added (1.5 ml serum-free DMEM containing 0.5 g/l MTT) and incubated in the dark at 37° C., 5% CO₂ for 2 hours. Tubes are centrifuged for 10 minutes at 2000 g and the supernatant removed. Isopropyl alcohol (1.5 ml) is added and constructs disintegrated using steel balls and a Minibead-beadbeater (Biospec, Bartlesville, Okla.). Tubes will be centrifuged at 2000 g for 10 minutes, and absorption measured in the supernatant at 570 nm.

2.3.4.3 GAG analysis-samples (n=4 per group and time point) are frozen, lyophilized, and digested for 15 hours at 56° C. with 1 mg/cm³ proteinase K solution in buffer (50 mM TRIS, 1 mM EDTA, 1 mM iodoacetamide, 10 μg/cm³ pepstatin-A) using 1 cm³ enzyme solution per 4-10 mg dry weight of the sample. GAG content is determined spectrophotometrically (Perkin Elmer, Oak Bridge IL) at 525 nm following binding to the dimethylmethylene blue dye.

2.3.4.4 RNA isolation and real-time Reverse Transcription Polymerase Chain Reaction (real time RT-PCR)—Fresh constructs (n=4 per group and time point) are transferred into 2 ml plastic tubes and 1.5 ml Trizol added. Constructs are disintegrated using steel balls and a Minibead-beadbeater (Biospec, Bartlesville, Okla.). Tubes are centrifuged at 12,000 g for 10 min and the supernatant transferred to a new tube. Chloroform (200 μl) is added to the solution and incubated for 5 minutes at room temperature. Tubes again are centrifuged at 12,000 g for 15 min and the upper aqueous phase transferred to a new tube. One volume of 70% ethanol (v/v) is added and applied to an RNeasy mini spin column (Quiagen, Hilden, Germany). The RNA is washed and eluted according to the manufacturer's protocol. The RNA samples are reverse transcribed in cDNA using oligo (dT)-selection according to the manufacturer's protocol (Superscript Preamplification System, Life Technologies, Gaithersburg, Md.). Collagen type II transcript levels can be quantified using the ABI Prism 7000 Real Time PCR system (Applied Biosystems, Foster City, Calif.). PCR reaction conditions are 2 min at 50° C., 10 min at 95° C., 50 cycles at 95° C. for, ISs, and 1 min at 60° C. The expression data is normalized to the expression of the housekeeping gene, glyceraldehyde-3-phosphate-dehydrogenase (GAPDH). Probes are labeled at the 5′ end with fluorescent dye FAM (VIC for GAPDH) and with the quencher dye TAMRA at the 3′ end. Primers and probes can be purchased from Applied Biosciences (assay on Demand).

2.3.4.5 Histology, immunohistochemistry and scanning electron microscopy (SEM)—For histology, constructs are fixed in neutral-buffered formalin (24 h at 4° C.), dehydrated in graded ethanol solutions, embedded in paraffin, bisected through the center and cut into 5 μm thick sections. To stain for GAG, sections are treated with eosin for 1 min, fast green for 5 min, and 0.2% aqueous safranin O solution for 5 min, rinsed with distilled water, dehydrated through xylene, mounted, and placed under a coverslip. To immunostain for type II collagen, a monoclonal antibody against type II collagen (2B1.5, dilution 1:100, Neomarkers, Fremont, Calif.) can be used. Paraffin embedded tissue sections are deparrafinized through a series of graded alcohols, and treated with protease II for 16 min. The primary antibody is added to each slide and the slide incubated for 32 minutes at room temperature in a humidified chamber. The secondary antibody (horseradish peroxidase) is applied and developed according to the manufacturer's protocol (BenchMark IHC staining module, Ventana, Tucson, Ariz.). Sections are counterstained using hematoxylin for 2 minutes. For SEM, constructs are bisected either en-face section or in cross-section and fixed for 12 h in the Karnovsky's fixative (2% paraformaldehyde, 2% glutaraldehyde in PBS). The constructs are subsequently fixed with 1% osmium tetroxide in 0.1 M phosphate buffer for 1 h, rinsed with PBS for 15 min. dehydrated using a series of graded alcohols and dried. Constructs can be gold-sputtered prior to evaluation.

### 2.2.5. Statistical Analysis

Statistical analysis of data was performed by one-way analysis of variance (ANOVA) and Tukey-Kramer procedure for post hoc comparison. Differences between the groups with p<0.05 were considered statistically significant.

### 2.2.2. Mechanical Tests

The mechanical properties in the wet state (never dried) are determined using standard tension tests and a custom-made confined compression chamber and an Instron Testing Machine 8511 with a 50 lb load cell.

The concept of the Bio-LOM scaffolds can be translated to micro-Bioreactor designs with multiple channels for nutrients and structural support. The growth of the BC lining is an example of a bioreactor. By varying grid patterns and z-direction pathways the micro-bioreactor begins to look more like a Bio-MEMS microfluidic device similar to DNA chip analyzers and other similar devices under development. The 3D fabrication of Bio-MEMS is complex using standard VLSI technologies. The laminated object manufacturing approach and/or the in-situ organic material growth promise growth opportunities for a multitude of cells.

### REFERENCES

The references cited herein and throughout the specification are incorporated by reference.
1. Freeman M. Adult Articular Cartilage. London: Grune&Stratton, 1973.
2. Huber M, Trattnig S, Lintner F. Anatomy, biochemistry, and physiology of particular cartilage. Invest Radiology 2000; 35:573-580.
3. Wilkins R J, Browning J A, Ellory J C. Surviving in a matrix: membrane transport in articular chondrocytes. J Mem Biol 2000; 177:95-108.
4. Rheumatology, A. C.o. Osteoarthritis. (www.rheumatology.org/patients/factsheet/oa.html, 2000).
5. Ma P X, Langer R. Morphology and mechanical function of long-term in vitro engineered cartilage. J Biomed Mat Res 1999; 44:217-221.
6. Suh J K F, Matthew H W T. Application of chitosan-based polysaccharide biomaterials in cartilage tissue engineering: a review. Biomaterials 2000; 21:2589-2598.
7. Schreiber R E, Dunkelman N S, Naughton G, Ratcliffe A. A method for tissue engineering of cartilage by cell seeding on bioresorbable scaffolds. In: Hunkler D, Prokop A, Chemington A D, Rajotte R V, Sefton M, editors. Bioartificial Organs II. Technology, Medicine and Materials. New York: New York Academy of Science, 1999. p. 398-404.
8. Sherwood J K, Riley S L, Palazzolo R, Brown S C, Monkhouse D C, Coates M, Griffith L G, Landeen L K, Ratcliffe A. A three-dimensional osteochondral composite scaffold for articular cartilage repair. Biomaterials 2002; 23:4739-4751.
9. Gugala Z, Gogolewski S. In vitro growth and activity of primary chondrocytes on a resorbable polylactide three-dimensional scaffold. J Biomed Mat Res 2000; 49:183-191.
10. Elisseeff J H, Lee A, Kleinman H K, Yamada Y. Biological esponse of chondrocytes to hydrogels. In: Sipe J D, Kelley C A, McNichol L A, editors. Reparative Medicine Growing Tissues and Organs. New York: N.Y. Academy of Science, 2002. Vol. 961, p. 118-122.
11. Brun P, Abatangelo G, Radice M, Zacchi V, Guidolin D, Gordini D D, Cortivo R. Chondrocyte aggregation and reorganization into three-dimensional scaffolds. J Biomed Mat Res 1999; 46:337-346.
12. Hutmacher D W. Scaffolds in tissue engineering bone and cartilage. Biomaterials
13. Lee K Y, Mooney D J. Hydrogels for tissue engineering. Chem Reviews 2001; 101:1869-1879.
14. Riesle J, Hollander A P, Langer R, Freed L E, Vunjak-Novakovic G. Collagen in tissue-engineered cartilage: Types, structure, and crosslinks. J Cell Biochem 1998; 71:313-327.
15. Grande D A, Halberstadt C, Naughton G, Schwartz R, Manji R. Evaluation of matrix scaffolds for tissue engineering of articular cartilage grafts. J Biomed Mat Res 1997; 34:211-220.
16. Seal B L, Otero T C, Panitch A. Polymeric biomaterials for tissue and organ regeneration. Mat Sci Eng: R 2001; 34:147-230.
17. Temenoff J S, Mikos A G. Review: tissue engineering for regeneration of articular cartilage. Biomaterials 2000;21:431-440.
18. Sachlos, E. and Czernuszka, J. T. Making tissue engineering scaffolds work. European Cells and Materials 2003; 5:29-40.
19. Mikos, A. G., et al. U.S. Pat. No. 5,514,378. Biocompatible polymer membranes and methods of preparation of three dimensional membrane structures; May 7, 1996.
20. Mikos, A. G., Temeoff, J. S. Formation of highly porous biodegrabadle scaffolds for tissue engineering. Electronic Journal of Biotechnology
21. Bhatia, S, and Chen, C. Tissue engineering at the micro-scale. Biomedical Microdevices 1999; 2:131-144.
22. Ciardelli, G., Chiono, V., et al. Innovative tissue engineering structures through advanced manufacturing technologies. Journal of Materials Science: Materials in Medicine 2003; 15:305-310.
23. Vozzi G., Flaim, C., et al. Fabrication of PLGA scaffold using soft lithography and microsyringe deposition. Biomaterials 2003; 24:2533-2540.
24. Vozzi G., Flaim, C., et al. Microfabricated PLGA scaffolds: a comparative study for application to tissue engineering. Materials Science and Engineering C 2002; 20:43-47.
25. Malda J., Woodfield, T. B. F., et al. The effect of PEGT/PBT scaffold architecture on the composition of tissue engineered cartilage. Biomaterials 2005; 26:63-72.
26. Woodfield, T. B. F., Malda, J., et al. Design of porous scaffolds for cartilage tissue engineering using a three-dimensional fiber-deposition technique. Biomaterials 2004; 25:4149-4161.
27. Hutmacher, D. W., Schantz, T., Zein, I. et al. Mechanical properties and cell cultural response of polycaprolactone scaffolds designed and fabricated via fused deposition modeling. Journal of Biomaterial Research 2001; 55:203-16.
28. Zein, I. Hutmacher, D. W., et al. Fused deposition modeling of novel scaffold architectures for tissue engineering applications. Biomaterials 2002; 23:1169-1185.
29. Svensson A, Nicklasson E, Harrah T, Panilaitis B. Kaplan D L, Brittberg M, and Gatenholm P, Bacterial cellulose as a potential scaffold for tissue engineering of cartilage. Biomaterials 2005; 26:419-431.
30. Meinel L, Hofmann S, Karageorgiou V, Kirker-Head C, McCool J, Gronowicz G, Zichner L, Langer R, Vunjak-Novakovic G, and KaplanDL, The inflammatory responses to silk films in vitro and in vivo. Biomaterials 2005; 26:147-155.
31. Naritomi T, Kouda T, Yano H, Yoshinaga F. Effect of lactate in bacterial cellulose production from fructose in continuous culture. J Ferm Bioeng, 1998; 85:89-95.
32. Tahara N, Tabuchi M, Watanabe K, Yano H, Morinaga Y, Yoshinaga F. Degree of polymerization of cellulose from Acetobacter xylinum decreased by cellulase produced by the strain. Biosci Biotechnol Biochem 1997; 61:1862-1865.
33. Miyamoto. T, Takahashi S, Ito H, Inagaki H, Noishiki Y. Tissue biocompatibility of cellulose and its derivatives. J Biomed Mater Res 1989; 23:125-133.
34. Ross P, Mayer R, Benziman M. Cellulose biosynthesis and function in bacteria. Microbio Rev 1991; 55:35-58.
35. Lee J W, Deng F, Yeomans W G, Allen A L, Gross R A, Kaplan D L. Direct incorporation of glucosamine and N-acetylglucosamine into exopolymers by Gluconacetobacter xylinus (=Acetobacter xylinum) ATCC 10245: production of chitosan-cellulose and chitin-cellulose exopolymers. App Environ Microbio 2001; 67:3970-3975.
36. Klemm D, Schumann D, Udhardt U, Marsch S. Bacterial synthesized cellulose—artificial blood vessels for microsurgery. Prog Polym Sci 2001; 26:1561-1603.
37. Xia, Y., Whitesides, G. Soft Lithography. Annual Reviews Material Science 1998; 28:153-84.

We claim:

1. A multilayer scaffold, comprising:
a. at least a first layer comprised of a polymer having a defined pattern of microchannels therein; and
b. at least a second layer comprised of a polymer having a defined pattern of microchannels therein;
wherein the first and second layer are joined together and the channels are connected for the circulation of fluid through the layers and growth of cellular material, and further wherein the scaffold is coated with bacterial cellulose.

2. The multilayer scaffold of claim 1, further comprising a mammalian cell.

3. The multilayer scaffold of claim 1, wherein the polymer of the first and second layer is a protein, polysaccharide, elastomer, or synthetic polymer.

4. The multilayer scaffold of claim 1, wherein the polymer of the first and second layer is polycoprolactone (PCL) and/or polylactide-co-glycolide (PLGA).

5. The multilayer scaffold of claim 1, wherein the layers are joined by lamination.

6. A method for producing a multilayer scaffold of claim 1 comprising:
a. providing at least a first layer comprised of a polymer having a defined pattern of microchannels therein;
b. providing at least a second layer comprised of a polymer having a defined pattern of microchannels therein;
c. joining the first and second layer such that channels are connected for the circulation of fluid through the layers; and
d. placing the joined layers in a growing bacterial culture for a sufficient period of time to allow the layers to be coated with bacterial cellulose.

7. The method of claim 6, further comprising contacting the scaffold with mammalian cells placed under appropriate conditions to allow the mammalian cells to proliferate on the scaffold.

8. The multilayer scaffold of claim 6, wherein the polymer of the first and second layer is a protein, polysaccharide, elastomer, or synthetic polymer.

9. The multilayer scaffold of claim 6, wherein the polymer of the first and second layer is polycoprolactone (PCL) and/or polylactide-co-glycolide (PLGA).

10. The scaffold or method of any preceding claim, wherein the mammalian cells include cells selected from the group consisting of hepatocytes, pancreatic Islet cells, fibroblasts, chondrocytes, osteoblasts, exocrine cells, cells of intestinal origin, bile duct cells, parathyroid cells, thyroid cells, cells of the adrenal-hypothalamic-pituitary axis, heart muscle cells, kidney epithelial cells, kidney tubular cells, kidney basement membrane cells, nerve cells, blood vessel cells, cells forming bone and cartilage, smooth muscle cells, skeletal muscle cells, oscular cells, integumentary cells, bone marrow cells, keratinocytes, pluripotent cells and stem cells and combinations thereof.
